(19) United States
(12) Patent Application Publication
(10) Pub. No.: US 2005/0209464 A1
(43) Pub. Date: Sep. 22, 2005

(54) Process for preparing optically active 1- (4-nitrophenyl) -3-pyrrolidinol derivatives, and chiral para-phenylenediamines containing a pyrrolidinyl group

(76) Inventors: Thierry Bordier, Tremblay en France (FR); Jinzhu Xu, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/995,559

(22) Filed: Nov. 24, 2004

Related U.S. Application Data
(60) Provisional application No. 60/531,966, filed on Dec. 24, 2003.

(30) Foreign Application Priority Data
Nov. 28, 2003 (FR) 03 50940

(51) Int. Cl. C07D 209/36

(52) U.S. Cl. 548/484

(57) ABSTRACT

A process for preparing an optically active 1-(4-nitrophenyl)-3-pyrrolidinol derivative corresponding to formula (I) below: 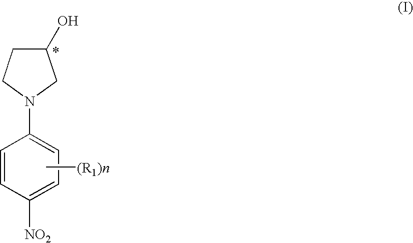
para-Phenylenediamine derivatives substituted by a chiral pyrrolidinyl group, of formula (II), and their addition salts: 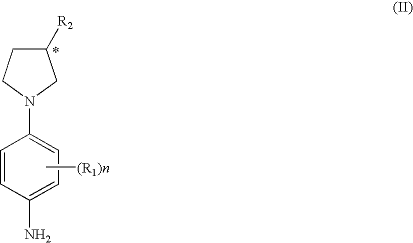 A dyeing composition comprising a para-phenylenediamine derivative of formula (II). A method of oxidation-dyeing keratin fibers which employs said composition. Multicompartment devices in which a first compartment contains the said dyeing composition and a second compartment contains an oxidizing agent.

### DESCRIPTION

The invention relates to a process for preparing optically active 1-(4-nitrophenyl)-3-pyrrolidinol derivatives.

The invention also relates to new chiral para-phenylenediamines containing a pyrrolidinyl group.

The optically active 1-(4-nitrophenyl)-3-pyrrolidinols correspond, for example, to the formula (I) below: 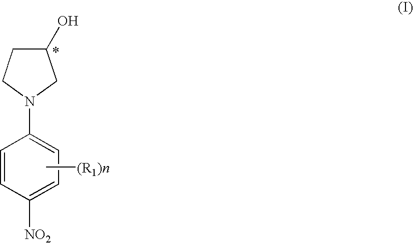
in which:
the carbon bearing the OH substituent is chiral (R or S);
n is between 0 and 4, with the proviso that when n is greater than or equal to 2 the radicals R₁ may be identical or different;
R₁ represents a halogen atom; a saturated or unsaturated, C₃-C₈ alicyclic, or C₁-C₈ linear or branched, aliphatic hydrocarbon chain, an aryl radical, an arylalkyl radical whose alkyl moiety is C₁-C₈, it being possible for one or more carbon atoms of the hydrocarbon chain and of the alkyl chain of the arylalkyl radical to be replaced by an oxygen, nitrogen, silicon or sulfur atom or by an SO₂ group, the radical R₁ not containing a peroxide linkage or diazo, nitro or nitroso radicals.

These compounds are important precursors for the synthesis of para-phenylenediamines containing a chiral pyrrolidinyl group which are substituted by a radical R₂ which corresponds to the formula (II) below: 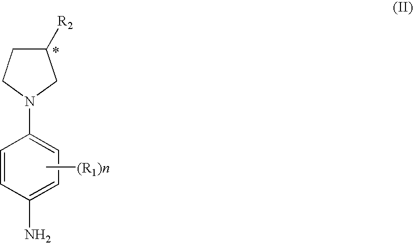
in which:
the carbon bearing the substituent R₂ is chiral (R or S);
n and R₁ are as already defined above; and
R₂ represents a cationic or noncationic nitrogen-containing radical.

The compounds of formula (II) are used as an oxidation base for dyeing and in dyeing kits and have the advantage of exhibiting a favorable toxicological profile.

Some compounds of formula (I), in which the carbon bearing the OH substituent on the pyrrolidine ring possesses the R configuration, are known and their synthesis has already been described; in contrast, the compounds of formula (I) having the S configuration are not known.

Thus document WO-A-01 68043 describes the preparation of the compound (IV) by reaction between 4-fluoronitrobenzene and (R)-3-pyrrolidinol, in accordance with scheme 1 below:

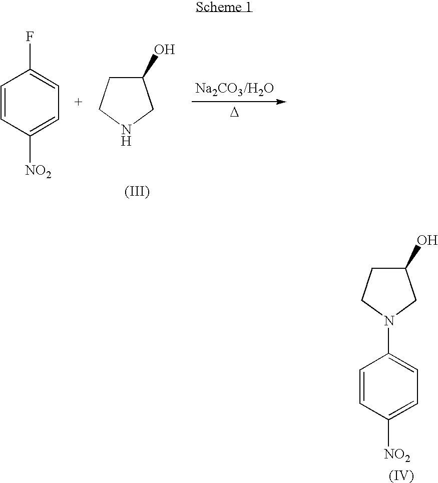

Chiral 3-pyrrolidinols (for example, of formula (III), of R or S configuration), which are the starting materials used in this type of synthesis, are expensive compounds, which are difficult to obtain in the case of the R configuration compounds or are even unavailable industrially in the case of the S configuration compounds.

A number of processes for synthesizing (R)- or (S)-3-pyrrolidinols are known. These processes are in particular the following:
1. Reduction with LiAlH₄ of an N-substituted (S)-3-hydroxypyrrolidone-2,5-dione, obtained by condensing L-malic acid with a primary amine, leading to the preparation of the (S)-3-pyrrolidinol. This process is described in EP-A-0 398 720; Synthetic Communication, 1985, 15(7), 587-98; and in Journal of Medecinal Chemistry, 1994, 37, 2138-44.
2. Use of chiral 1,2,4-butanetriol as starting material, going via a cyclosulfinylalkyl halide derivative. This process is described in WO-A-00 15610.
3. Enzymatic or chemical resolution of racemic 3-pyrrolidinols. These processes are described in JP-A-07 188124 and WO-A-09 503421.
4. Decarboxylation of trans-4-hydroxy-L-proline for the synthesis of the R isomer, according to scheme 2 illustrated below. In a number of documents this process is described, with different protocols for isolating the end product.

The document Synthetic Communication, 1993, 23, 2691-9 describes the isolation of the neutral form by distillation under a reduced atmosphere.

Document WO-A-97 43256 describes the isolation of the hydrochloride form by precipitation with hydrochloric acid.

The document Journal of Chemical Society Perkin Translation I, 1993, 1421 reports the isolation of the decarboxylation product by a precipitation with maleic acid.

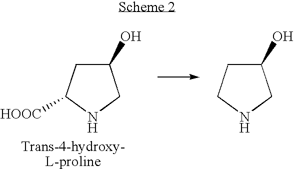

All of the abovementioned processes, with the exception of the fourth, employ long and inconvenient synthesis processes, with expensive reactants or starting materials for some of them.

The decarboxylation of trans-4-hydroxy-L-proline to (R)-3-pyrrolidinol (process 4) is the most direct process. However, isolation of the product requires either an inconvenient distillation or precipitation with an acid. Industrial application may prove inconvenient. Moreover, this process is limited to the preparation of the R configuration isomer.

In the light of the above there exists a need for a process for preparing chiral, optically active 1-(4-nitrophenyl)-3-pyrrolidinol derivatives, corresponding in particular to the formula (I) above, which is easy to implement, reliable, safe and reproducible and which includes only a limited number of simple steps.

There is still a need for a process for preparing these compounds which uses starting materials or primary products which are readily available and of low cost and which allows the final 1-(4-nitrophenyl)-3-pyrrolidinol derivatives to be obtained with a high yield.

There is in particular a need for a process of this kind which avoids the use, as starting materials, of 3-pyrrolidinols, which are expensive compounds of limited or zero availability which are extremely long and difficult to synthesize.

The aim of the present invention is to provide a process for preparing optically active 1-(4-nitrophenyl)-3-pyrrolidinol derivatives which meets needs including those set out above.

The aim of the present invention is also to provide a process for preparing optically active 1-(4-nitrophenyl)-3-pyrrolidinols which do not exhibit the drawbacks, defects, limitations and disadvantages of the prior art processes and which solves the problems of the prior art processes.

This aim and others are achieved in accordance with the invention, in a first variant, by a synthesis process for preparing optically active 1-(4-nitrophenyl)-3-pyrrolidinol derivatives corresponding to the formula (I) below: 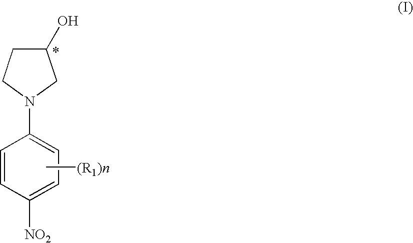
in which:
the carbon bearing the OH substituent on the pyrrolidine ring is chiral, and possesses the (R) or (S) configuration;
n is an integer of 0 to 4, with the proviso that when n is greater than or equal to 2 the radicals R₁ may be identical or different;
R₁ represents a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl moiety is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ not containing a peroxide linkage nor diazo, nitro or nitroso radicals;
   wherein the following successive steps are carried out:
a) trans-4-hydroxy-L-proline is decarboxylated: 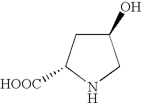
   to give (R)-3-pyrrolidinol: 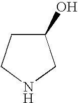
   b) without isolation, the resulting (R)-3-pyrrolidinol formed is reacted with a 4-halonitrobenzene of formula (V): 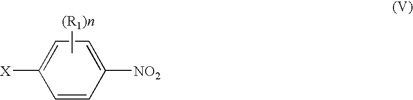
   in which X represents an F, Cl, Br, or I atom, and R₁ is as already defined above for the formula (I), thereby giving the compound of formula (I) in which the carbon bearing the —OH substituent on the pyrrolidine ring possesses the (R) configuration (compound I_{R}): 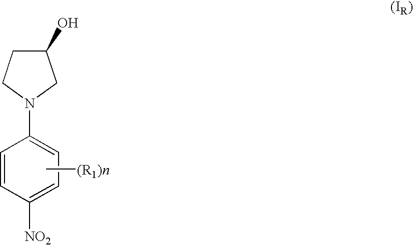
   c) optionally the configuration of the compound of formula (I_{R}) is inverted, thereby giving the compound of formula (I_{S}):

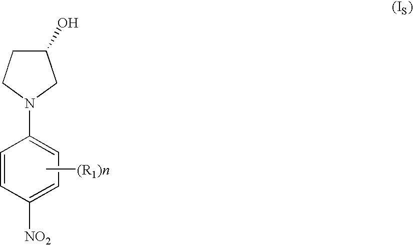

According to a second variant of the process of the invention, the aforementioned aims and others are also achieved by a process for preparing an optically active 1-(4-nitrophenyl)-3-pyrrolidinol derivative of the formula (I): 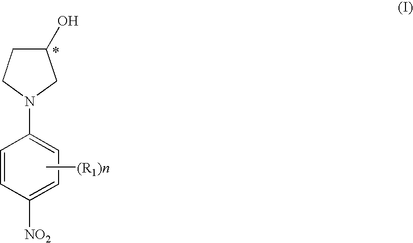
in which:
the carbon bearing the OH substituent on the pyrrolidine ring is chiral, and possesses the (R) or (S) configuration;
n an integer of 0 to 4, with the proviso that when n is greater than or equal to 2 the radicals R₁ may be identical or different;
R₁ represents a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl moiety is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ not containing a peroxide linkage nor diazo, nitro or nitroso radicals;
   wherein the following successive steps are carried out:
a′) trans-4-hydroxy-L-proline: 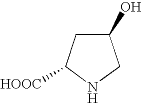
   is condensed with a 4-halonitrobenzene of formula (V): 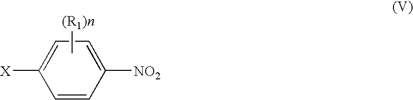
   in which X represents an F, Cl, Br or I atom and R₁ is as already defined above for the formula (I), to give a compound of formula (VI): 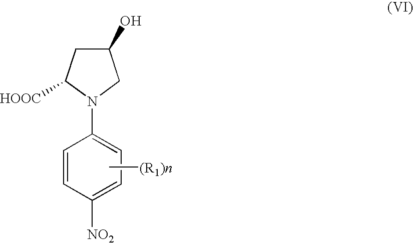
   b′) the compound of formula (VI) is decarboxylated to give the compound of formula (I) in which the carbon bearing the OH substituent on the pyrrolidine ring possesses the (R) configuration (compound I_{R}): 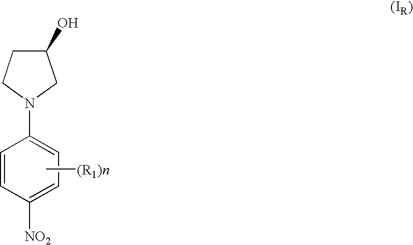
   c′) optionally the configuration of the compound of formula (I_{R}) is inverted, thereby giving a compound of formula (I_{S}):

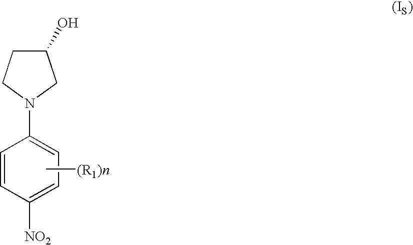

The process according to the invention, in both its first and its second variant, employs primary products which are readily available industrially and of low cost, since it essentially involves, on the one hand, trans-4-hydroxy-L-proline and, on the other hand, 4-halonitrobenzenes, which are common products that are relatively inexpensive.

The process according to the invention does not employ, in contrast to the prior art processes, the use of chiral 3-pyrolidinols as starting material, and thus avoids one of the major drawbacks of the prior art processes.

It should be noted that in the first variant of the process according to the invention an (R)-3-pyrrolidinol is formed, but this is an intermediate and not a primary product, and it is not necessary to isolate this compound, which can be reacted directly with the 4-halonitrobenzene.

In the second variant of the process according to the invention there is also no need to prepare the (R)-3-pyrrolidinol beforehand.

The process according to the invention, in both its first and its second variant, is a simple and reliable process which features a limited number of simple steps which are easy to implement, and the overall yield of the process is high.

The process according to the invention provides access both to the compound in which the carbon bearing the OH substituent on the pyrrolidine ring is in the (R) configuration and to the compound in which this carbon atom is in the (S) configuration.

The invention relates, moreover, to new para-phenylenediamine derivatives which are substituted by a chiral pyrrolidinyl group, of formula (II), and their addition salts: 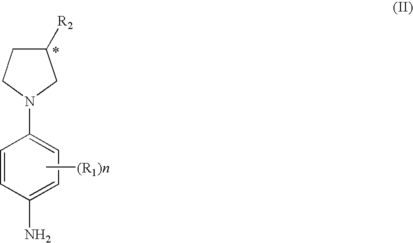
in which:
the carbon atom which bears the R₂ substituent on the pyrrolidine ring is chiral and possesses the (R) configuration (derivative (II_{R})) or (S) configuration (derivative (II_{S}));
R₁ and n are as already defined above for the derivatives of formula (I);
R₂ represents a cationic or noncationic nitrogen-containing radical.

The invention further provides a dyeing composition comprising, in a medium appropriate for the dyeing of keratin fibers, at least one para-phenylenediamine derivative of formula (II), in an (R) or (S) configuration, as oxidation base.

The invention likewise relates to a method of oxidation-dyeing keratin fibers, in which a dyeing composition as defined above is applied to the fibers in the presence of an oxidizing agent for a time sufficient for the desired coloration to develop.

The present invention also provides a multicompartment device in which a first compartment contains a dyeing composition as defined above and a second compartment contains an oxidizing agent.

The invention provides still further for the use of this composition for dyeing keratin fibers and a method of dyeing keratin fibers, especially human keratin fibers such as the hair, which employs the composition of the present invention.

The para-phenylenediamine (PPD) derivatives substituted by a chiral pyrrolidinyl group, of the formula (II), obtained from the compounds of formula (I) provided by the invention, are used as an oxidation base to obtain coloration of chromatic keratin fibers that is powerful, unselective and persistent.

The invention will now be described in detail in the text below.

In the context of the invention an aliphatic hydrocarbon chain is a linear or branched chain which may contain unsaturations of the alkene or alkyne type. An alicyclic hydrocarbon chain is a saturated or unsaturated cyclic chain which does not contain an aromatic cyclic structure.

When the chain is interrupted by an oxygen, sulfur, nitrogen or silicon atom Y or by SO₂ the result, for example, is a unit CH₂—Y—CH₂.

In the formula (I) n can be 0 and in that case the benzene ring does not bear any substituent. In the contrary case (n other than 0), and by way of example, R₁ may be a chlorine atom or a methyl, ethyl, isopropyl, hydroxymethyl methoxymethyl, hydroxyethyl, 2-hydroxyethyl, 3-hydroxypropyl, 1,2-dihydroxyethyl, methoxy, ethoxy, 2-hydroxyethyloxy or phenyl radical.

In the formula (I), when n is other than 0 and is for example 1, R₁ is preferably a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl chain is C₁-C₈ alicyclic, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ containing no peroxide linkage and no diazo, nitro or nitroso radicals.

Preferably (n being other than 0 and being, for example, 1) R₁ is selected from chlorine, bromine, a C₁-C₆, advantageously C₁-C₄, alkyl, C₁-C₄ hydroxyalkyl, C₁-C₄ alkoxy, (C₁-C₄)-alkoxy-(C₁-C₄) alkyl, for example methoxymethyl, or C₁-C₄ hydroxyalkoxy radical.

By way of example, R₁ is selected from methyl, hydroxymethyl, 2-hydroxyethyl, 1,2-dihydroxy-ethyl, methoxy, isopropyloxy and 2-hydroxyethoxy radicals.

The various steps of the process of the invention according to its first variant (route A) or according to its second variant (route B) are illustrated in simplified form in scheme 3 below:

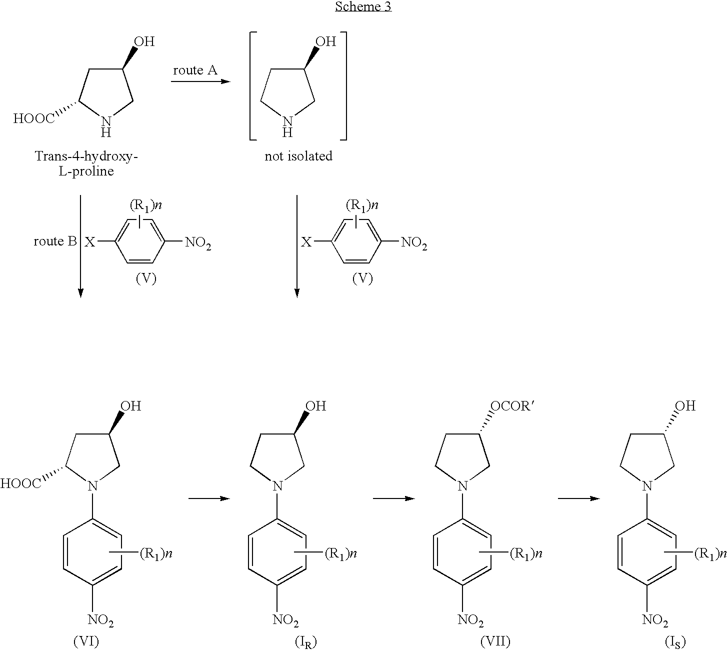

In the first variant of the process according to the invention, first of all in step a), the trans-4-hydroxy-L-proline is decarboxylated (see scheme 3) to give the (R)-3-pyrrolidinol.

This decarboxylation is carried out under appropriate conditions, which are known to the skilled worker in this field of the art.

The decarboxylation may thus be carried out as follows: the trans-4-hydroxy-L-proline is heated in a solvent in the presence of a catalyst such as cyclohexen-1-one. The temperature is generally between 100 and 180° C. The reaction is generally carried out in the presence of a solvent, which is commonly selected from dimethylformamide, dimethyl sulfoxide, N-methyl-pyrolidinone or certain high boiling point alcohols such as cyclohexanol, pentanol and butanol.

At the end of this step a), the (R)-3-pyrrolidinol formed is not separated, which is to say that it is left in the vessel or reactor in which step a) of the process has taken place and a 4-halonitrobenzene of formula (V) in which X represents an F, Cl, Br or I atom and R₁ is as already defined above is introduced into said vessel or reactor in order to react with the (R)-3-pyrrolidinol.

The reaction between the (R)-3-pyrrolidinol and the 4-halonitrobenzene (V) is generally carried out as follows: the 4-halonitrobenzene and a base are introduced into the mixture containing the (R)-3-pyrrolidinol and the system is heated. The reaction temperature is more particularly between 75° C. and 160° C. The base is selected in particular from sodium carbonate, sodium bicarbonate, potassium carbonate, potassium bicarbonate, sodium acetate and potassium acetate.

In the second variant of the process according to the invention (route B in scheme 3) the first step a′) of the process consists in condensing the trans-4-hydroxy-L-proline, which is to say the same starting compound as in the first variant of the process of the invention, with a 4-halonitrobenzene of formula (V) to form a compound of formula (VI).

The general conditions of this condensation reaction are as follows: the 4-halonitrobenzene and the trans-4-hydroxy-L-proline are heated in a solvent in the presence of a base. The temperature is customarily between 75 and 180° C. The solvent is selected more particularly from dimethylformamide, dimethyl sulfoxide, N-methylpyrolidinone, water and alcohols such as propanol or isobutanol. The base is preferably selected from sodium carbonate, sodium bicarbonate, potassium carbonate, potassium bicarbonate, sodium acetate and potassium acetate.

The compound of formula (VI) is subsequently decarboxylated (step b′)) to give the compound of formula (I) in which the carbon bearing the OH substituent on the pyrrolidine ring possesses the (R) configuration (compound I_{R}).

The decarboxylation reaction of the compound (VI) is generally carried out as follows: a mixture of the compound of the formula (VI) is heated with a catalyst such as cyclohexen-1-one in a solvent. The temperature is generally between 100 and 180° C. The solvent is selected more particularly from dimethylformamide, dimethyl sulfoxide, N-methyl-pyrolidinone and high boiling point alcohols such as cyclohexanol, butanol and pentanol, in particular.

At the end of step b) of the first variant of the process of the invention, or at the end of step b′) of the second variant of the process of the invention, it is possible optionally, if the desire is to obtain the compound of formula (I) in which the carbon bearing the OH substituent on the pyrrolidine ring, which posseses the (S) configuration (compound I_{S}), to carry out inversion of configuration of the compound of formula (I_{R}) obtanined in step b) or b′) in an optional step c) or c′) respectively.

This inversion of the configuration may be carried out by any configuration inversion process known in this technical field.

It will be possible, for example, to perform a Mitsunobu reaction (see documents Bulletin of the Chemical Society of Japan, 1967, volume 40, 2380, Australian Journal of Chemistry, 1988, volume 41, 1835, and Organic Preparations and Procedures International 1996, volume 28, 127) on the compound of formula (I_{R}) to give a compound of formula (VII): 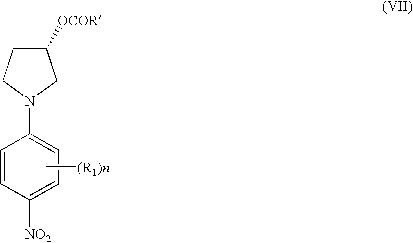
in which R₁ and n are as already defined above and in which R′ represents a hydrogen atom, a C₁-C₆ hydrocarbon radical or a C₆-C₈ aryl radical. In the compound of formula (VII) the carbon atom bearing the group OCOR′ is in the (S) configuration.

The general conditions of the Mitsunobu reaction are as follows: triphenylphosphine and a carboxylic acid, R′COOH, are added to a solution containing the compound of formula (I_{R}). Then diethyl azodicarbonate (EtOOCN═NCOOEt) is added. The product formed, of formula (VII), is subsequently isolated using conventional techniques, filtration or extraction for example. The carboxylic acid R′COOH is selected in particular from acetic acid, benzoic acid and formic acid. The reaction solvent is generally an ethereal solvent, such as tetrahydrofuran, diethyl ether or methyl tert-butyl ether.

The compound of formula (VII) is subsequently hydrolyzed under the following general conditions: the compound of formula (VII) is treated with a base selected from alkali metal hydroxides such as sodium hydroxide, potassium hydroxide or lithium hydroxide to give a compound of formula (I_{S}), which is isolated using conventional techniques.

The compounds of formula (I), of (R) or (S) configuration, are precursors for the synthesis of the para-phenylenediamines of formula (II), which bear a chiral pyrrolidine group and are substituted by a radical R₂.

This synthesis is generally carried out as follows:
a) the compound of formula (I), (IR) or (IS) is activated to form, respectively, a compound of formula (III), (IIIR) or (IIIS): 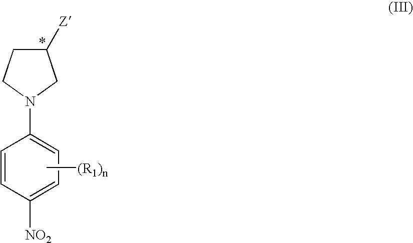
in which R₁ and n are as already defined above and Z′ represents a leaving group;
b) the compound of formula (III), (IIIR) or (IIIS) is reacted with a primary, secondary or tertiary amine or with a compound bearing an aromatic nitrogen-containing heterocycle
to form a compound of formula (IV), with inversion of configuration (IVR) or (IVS): 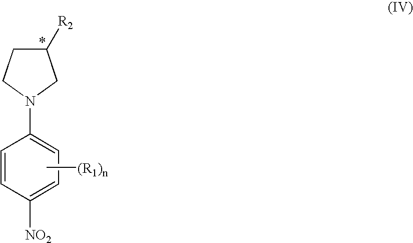
c) the NO₂ group is converted into an amine function by hydrogenation, to form the compound of formula (II), (II_{R}) or (II_{S}): 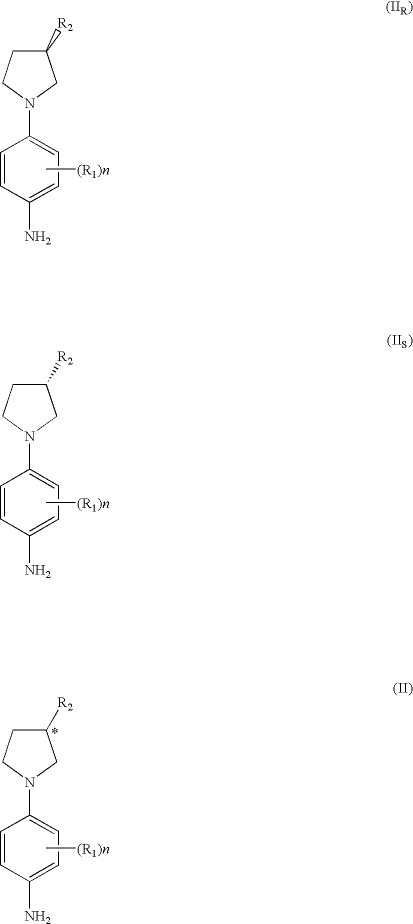
R₁ and n are as already defined (including the preferred definitions) above.

Also, as indicated earlier on, the radical R₂ represents a cationic or noncationic nitrogen-containing radical.

If R₂ is a cationic nitrogen-containing radical it represents more particularly an onium radical such as, for example, an ammonium, imidazolium or pyridinium radical.

If R₂ is a noncationic nitrogen-containing radical it represents more particularly a primary (—NH₂), secondary (—NHR) or tertiary (—NR₂) amine radical in which the RS, which are identical or different, represent a saturated or unsaturated, linear or branched C₁-C₂₂ aliphatic radical, preferably a C₁-C₂₂ alkyl radical; a saturated or unsaturated C₃-C₈ alicyclic radical; a C₁-C₂₂, preferably C₁-C₆, monohydroxyalkyl radical; a C₂-C₂₂, preferably C₂-C₆, polyhydroxyalkyl radical; a (C₁-C₆) alkoxy-(C₁-C₂₂, preferably C₁-C₆) alkyl radical; an aryl radical; an arylalkyl radical whose alkyl moiety is C₁-C₆, such as the benzyl radical, for example; a amido (C₁-C₆) alkyl radical; a tri-(C₁-C₆)alkyl-silane —(C₁-C₆) alkyl radical; a C₁-C₆ aminoalkyl radical; or a C₁-C₆ aminoalkyl radical whose amine is mono- or di-substituted by a C₁-C₄ alkyl, (C₁-C₆)alkyl-carbonyl, amido or (C₁-C₆)alkyl-sulfonyl radical.

Furthermore, the radicals R may form in pairs, together with the nitrogen atom to which they are attached, a saturated carbon ring containing 3 to 9 members, preferably 4, 5, 6, 7 or 8 members, which may contain one or more heteroatoms. As examples of such rings mention may be made of azetidine, pyrrolidine, piperidine, piperazine, morpholine rings, it being possible for said heterocycle to be substituted by a halogen atom, a hydroxyl radical, a C₁-C₆ alkyl radical, a C₁-C₆ monohydroxyalkyl radical, a C₂-C₆ poly-hydroxyalkyl radical, a C₁-C₆ alkoxy radical, a tri-(C₁-C₆)alkyl-silane-(C₁-C₆) alkyl radical, an amido radical, a carboxyl radical, a (C₁-C₆)alkyl-carbonyl radical, a thio radical (—SH), a C₁-C₆ thioalkyl radical (—R—SH), a (C₁-C₆)alkyl-thio radical, an amino radical, an amino radical mono- or di-substituted by a (C₁-C₆)alkyl, (C₁-C₆)alkyl-carbonyl, amido or (C₁-C₆)alkyl-sulfonyl radical.

In accordance with another embodiment R₂ is a radical derived from amino-guanidine (of formula —NH—NH—C(NH₂)═NH).

If the radical R₂ of the formula (II) is an onium radical Z it corresponds in a first embodiment to the formula (VIII): 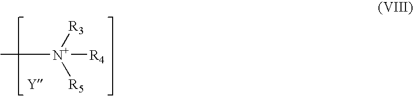
in which:
R₃, R₄ and R₅, taken separately, identical or different, represent a hydrogen atom, a saturated or unsaturated, linear or branched C₁-C₂₂ aliphatic radical, preferably a C₁-C₂₂ alkyl radical; a saturated or unsaturated C₃-C₈ alicyclic radical; a C₁-C₂₂, preferably C₁-C₆, monohydroxyalkyl radical; C₂-C₂₂, preferably C₂-C₆, polyhydroxyalkyl radical; a (C₁-C₆)alkoxy-(C₁-C₂₂, preferably C₁-C₆) alkyl radical; an aryl radical; an arylalkyl radical whose alkyl moiety is C₁-C₆, such as the benzyl radical, for example; a amido (C₁-C₆) alkyl radical; a tri-(C₁-C₆)alkyl-silane-(C₁-C₆) alkyl radical; a C₁-C₆ aminoalkyl radical; a C₁-C₆ aminoalkyl radical whose amine is mono- or di-substituted by a C₁-C₄ alkyl, (C₁-C₆)alkyl-carbonyl, amido or (C₁-C₆)alkyl-sulfonyl radical; or
R₃, R₄ and R₅ together, in pairs, form, with the nitrogen atom to which they are attached, a saturated carbon ring containing 3 to 9 members, preferably 4, 5, 6, 7 or 8 members, which may contain one or more heteroatoms; as examples of this ring mention may be made of azetidine, pyrrolidine, piperidine, piperazine or morpholine rings, it being possible for said cationic heterocycle to be substituted by a halogen atom, a hydroxyl radical, a C₁-C₆ alkyl radical, a C₁-C₆ monohydroxyalkyl radical, a C₂-C₆ polyhydroxyalkyl radical, a alkoxy radical, a C₁-C₆ tri-(C₁-C₆)alkylsilane-(C₁-C₆) alkyl radical, an amido radical, a carboxyl radical, a (C₁-C₆)alkyl-carbonyl radical, a thio radical (—SH), a C₁-C₆ thioalkyl radical (—R—SH), a (C₁-C₆)alkyl-thio radical, an amino radical, or an amino radical mono- or di-substituted by a (C₁-C₆)alkyl, (C₁-C₆)alkyl-carbonyl, amido or (C₁-C₆)alkyl-sulfonyl radical;
Y″ is a counterion.

In the formula (VIII), according to a more particular embodiment, R₃, R₄ and R₅ separately are selected preferably from a C₁-C₆ alkyl radical, a C₁-C₄ monohydroxyalkyl radical, a C₂-C₄ polyhydroxyalkyl radical, a (C₁-C₆)alkoxy-(C₁-C₄) alkyl radical, a amido (C₁-C₆) alkyl radical, a tri-(C₁-C₆)alkyl-silane-(C₁-C₆) alkyl radical, or R₃ together with R₄ forms an azetidine ring, a pyrrolidine, piperidine, piperazine or morpholine ring, R₅ being selected in this case from a C₁-C₆ alkyl radical; a C₁-C₆ monohydroxyalkyl radical; a C₂-C₆ polyhydroxyalkyl radical; a C₁-C₆ aminoalkyl radical; an aminoalkyl radical mono- or di-substituted by a (C₁-C₆)alkyl, (C₁-C₆)alkyl-carbonyl, amido or (C₁-C₆)alkyl-sulfonyl radical; a carbamyl-(C₁-C₆) alkyl radical; a tri-(C₁-C₆)alkyl-silane-(C₁-C₆) alkyl radical; a (C₁-C₆)alkyl-carboxy-(C₁-C₆)alkyl radical; a (C₁-C₆)alkyl-carbonyl-(C₁-C₆) alkyl radical; or a N-(C₁-C₆)alkyl-carbamyl-(C₁-C₆) alkyl radical.

When the radical R₂ corresponds to the formula (VIII) it is preferably a trialkylammonium radical whose alkyl radicals may be substituted.

According to a second embodiment, the radical R₂ represents an onium radical Z corresponding to the formula (IX): 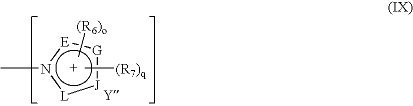
in which:
the ring members E, G, J and L, which are identical or different, represent a carbon, oxygen, sulfur or nitrogen atom, to form a pyrrole, pyrazole, imidazole, triazole, oxazole, isooxazole, thiazole or isothiazole ring;
q is an integer between 0 and 4 inclusive;
o is an integer between 0 and 3 inclusive;
q+o is an integer between 0 and 4 inclusive;
R₇s, which are identical or different, represent a hydroxyl radical, a C₁-C₆ alkyl radical, a C₁-C₆ monohydroxyalkyl radical, a C₂-C₆ polyhydroxyalkyl radical, a C₁-C₆ alkoxy radical, or a tri(C₁-C₆)alkyl-silane-(C₁-C₆) alkyl radical; with the proviso that the radicals R₇ are borne by a carbon atom;
   R₆, identical or different, represents a C₁-C₆ alkyl radical, a C₁-C₆ monohydroxyalkyl radical, a C₂-C₆ polyhydroxyalkyl radical, a tri(C₁-C₆)alkyl-silane-(C₁-C₆) alkyl radical or a benzyl radical; with the proviso that the radicals R₆ are borne by a nitrogen;
Y″ is a counterion.

By way of example the ring members E, G, J and L can form a pyrrole, imidazole, pyrazole, oxazole, thiazole or triazole ring, preferably an imidazole ring.

According to a third embodiment, R₂ represents the onium radical Z corresponding to the formula (X): 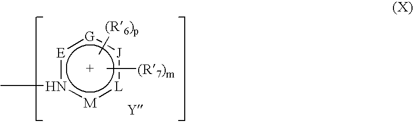
in which:
the ring members E, G, J, L and M, which are identical or different, represent a carbon, oxygen, sulfur or nitrogen atom, to form a ring selected from pyridine, pyrimidine, pyrazine, triazine and pyridazine rings;
p is an integer between 0 and 3 inclusive;
m is an integer between 0 and 5 inclusive;
p+m is an integer between 0 and 5 inclusive;
R′₇s, which are identical or different, represent a hydroxyl radical, a C₁-C₆ alkyl radical, a C₁-C₆ monohydroxyalkyl radical, a C₂-C₆ polyhydroxyalkyl radical, a C₁-C₆ alkoxy radical, or a tri (C₁-C₆)alkyl-silane-(C₁-C₆) alkyl radical; with the proviso that the radicals R′₇ are borne by a carbon atom;
R′₆, identical or different, represents a C₁-C₆ alkyl radical, a C₁-C₆ monohydroxyalkyl radical, a C₂-C₆ polyhydroxyalkyl radical, a tri(C₁-C₆)alkyl-silane-(C₁-C₆) alkyl radical, a (C₁-C₆) alkoxy-(C₁-C₆) alkyl radical, or a benzyl radical; with the proviso that the radicals R₁₆ are borne by a nitrogen;
Y″ is a counterion.

Preferably the ring members E, G, J, L and M form, with the nitrogen of the ring, a pyridine or pyrimidine ring.

Preferably R′₇ and R′₆, and also R₇ and R₆, are alkyl radicals which may be substituted.

In the context of the invention the counterion (Y″) may be selected from a halogen atom such as bromine, chlorine, fluorine or iodine, a hydroxide, a citrate, a succinate, a tartrate, a lactate, a tosylate, a mesylate, a benzenesulfonate, an acetate, a hydrogensulfate or a C₁-C₆ alkylsulfate such as, for example, methylsulfate or ethylsulfate.

Prferably the counterion (Y″) is selected from a halogen atom such as bromine, chlorine or iodine, a tosylate, a mesylate, a benzenesulfonate or a C₁-C₆ alkylsulfate such as, for example, methylsulfate or ethylsulfate.

Examples of compounds of formula (II) which may be prepared by the process of the invention are the following:

| Nomenclature | Structures |
|---|---|
| 1-[(3R or 3S)-1-(4- Aminophenyl)pyrrolidin-3- yl]-3-methyl-1H-imidazol-3- ium chloride, hydrochloride | 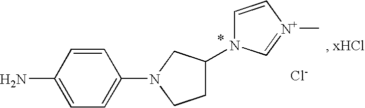 |
| [(3R or 3S)-1-(4- Aminophenyl) pyrrolidin-3- yl]-trimethyl-ammonium chloride, hydrochloride | 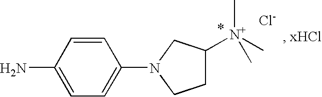 |
| 1-(4-Aminophenyl)- pyrrolidin-3(S or R)-amine dihydrochloride | 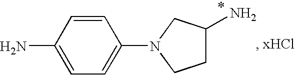 |
| 1-(4-Aminophenyl)-N- methylpyrrolidin-3(s or R)- amine dihydrochloride | 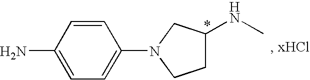 |
| 1-(4-Aminophenyl)-N,N- dimethylpyrrolidin-3(S or R)-amine dihydrochloride | 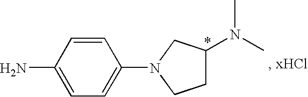 |
| 1-(4-Amino-3-methylphenyl)- pyrrolidin-3(S or R)-amine dihydrochloride | 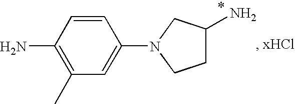 |
| [(3S or 3R)-1-(4-Amino-3- methylphenyl)-pyrrolidin-3- yl]-trimethyl-ammonium chloride, hydrochloride | 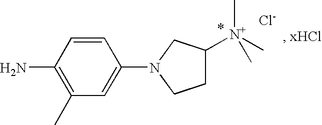 |
| [(3S or 3R)-1-(4-Amino-3- methylphenyl)-pyrrolidin-3- yl]-3-methyl-1H-imidazol-3- ium chloride, hydrochloride | 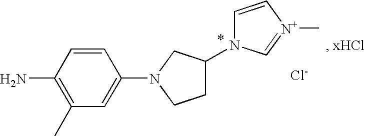 |

The dyeing composition of the present invention comprises, in a medium appropriate for dyeing keratin fibers, especially human keratin fibers such as human hair, as oxidation base, a derivative of formula (II), in the (R) or (S) configuration, as defined above.

The oxidation base or bases of the invention are generally each present in an amount of between 0.001 to 10% by weight approximately of the total weight of the dyeing composition, preferably between 0.005 and 6% by weight.

The dyeing composition of the invention may further comprise one or more couplers which are conventionally used for the dyeing of keratin fibers. Among these couplers mention may be made in particular of meta-phenylenediamines, meta-aminophenols, meta-diphenols, naphthalenic couplers, heterocyclic couplers and their addition salts.

By way of example mention may be made of 2-methyl-5-aminophenol, 5-N-(β-hydroxyethyl)amino-2-methylphenol, 6-chloro-2-methyl-5-aminophenol, 3-amino-phenol, 1,3-dihydroxybenzene, 1,3-dihydroxy-2-methyl-benzene, 4-chloro-1,3-dihydroxybenzene, 2,4-diamino-1-(β-hydroxyethyloxy)benzene, 2-amino-4-(β-hydroxyethyl-amino)-1-methoxybenzene, 1,3-diaminobenzene, 1,3-bis-(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxy-benzomorpholine 3,5-diamino-2,6-dimethoxypyridine, 1-N-(β-hydroxyethyl)amino-3,4-methylenedioxybenzene, 2,6-bis(β-hydroxyethylamino)toluene and their addition salts with an acid.

In the composition of the present invention the coupler or couplers are each present generally in an amount of between 0.001 and 10% by weight approximately of the total weight of the dyeing composition, preferably between 0.005 and 6%.

The composition of the present invention may further comprise one or more additional oxidation bases conventionally used in oxidation dyeing, other than those described above.

The amount of each of the additional oxidation bases is generally between 0.001 and 10% by weight approximately of the total weight of the dyeing composition.

By way of example these additional oxidation bases are selected from para-phenylenediamines other than those described above, bis-phenylalkylenediamines, para-aminophenols, bis-para-aminophenols, ortho-amino-phenols, heterocyclic bases and their addition salts.

Among the para-phenylenediamines mention may be made, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloro-aniline, 2-β-hydroxyethyl-para-phenylenediamine, 2-fluoro-para-phenylenediamine, 2-isopropyl-para-phenylene-diamine, N-(β-hydroxypropyl)-para-phenylene-diamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxy-propyl)-para-phenylenediamine, N-(4′-aminophenyl)-para-phenylenediamine, N-phenyl-para-phenylenediamine, 2-β-hydroxyethyloxy-para-phenylenediamine, 2-β-acetyl-aminoethyloxy-para-phenylenediamine, N-(β-methoxy-ethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine and their addition salts with an acid.

Among the abovementioned para-phenylenediamines para-phenylenediamine, para-tolylenediamine, 2-iso-propyl-para-phenylenediamine, 2-β-hydroxyethyl-para-phenylenediamine, 2-β-hydroxyethyloxy-para-phenylene-diamine, 2,6-dimethyl-para-phenylenediamine, 2,6-diethyl-para-phenylenediamine, 2,3-dimethyl-para-phenylenediamine, N,N-bis-(β-hydroxyethyl)-para-phenylenediamine, 4-N,N-bis(β-hydroxyethyl)amino-2-methylaniline, 4-N,N-bis-(β-hydroxyethyl)amino-2-chloroaniline, 2-β-hydroxyethyl-para-phenylenediamine, 2-fluoropara-phenylenediamine, 2-isopropyl-para-phenylenediamine, N-(β-hydroxyethyl)-para-phenylenediamine, 2-hydroxymethyl-para-phenylenediamine, N,N-dimethyl-3-methyl-para-phenylenediamine, N,N-(ethyl-β-hydroxy-ethyl)-para-phenylenediamine, N-(β,γ-dihydroxopropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylamino-ethyloxy-paraphenylenediamine, N═(β-methoxyethyl)-paraphenylenediamine, 4-aminophenylpyrrolidine, 2-thienylpara-phenylenediamine, 2-β-hydroxyethylamino-5-amino-toluene, 3-hydroxy-1-(4′-aminophenyl)pyrrolidine and their addition salts with an acid are preferred.

Among the abovementioned para-phenylenediamines para-phenylenediamine, paratolylenediamine, 2-isopropyl para-phenylenediamine, 2-β-hydroxyethyl-para-phenylene-diamine, 2-β-hydroxyethyloxy-para-phenylene-diamine, 2,6-dimethyl-para-phenylenediamine, 2,6-diethyl-para-phenylenediamine, 2,3-dimethyl-para-phenylenediamine, N,N-bis-(β-hydroxyethyl)-para-phenylenediamine, 2-chloro-para-phenylenediamine, 2-β-acetyl-aminoethyloxy-para-phenylenediamine and their addition salts with an acid are particularly preferred.

Among the bis-phenylalkylenediamines mention may be made, by way of example, of N,N′-bis(β-hydroxy-ethyl)-N,N′-bis(4′-aminophenyl)-1,3-diaminopropanol, N,N′-bis(β-hydroxyethyl)-N,N′-bis-(4′-amino-phenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methyl-aminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane and their addition salts with an acid.

Among the para-aminophenols mention may be made, by way of example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol and their addition salts with an acid.

Among the ortho-aminophenols mention may be made, by way of example, of 2-aminophenol, 2-amino-5-methylphenol, 2-amino-6-methylphenol, 5-acetamido-2-aminophenol and their addition salts with an acid.

Among the heterocyclic bases mention may be made, by way of example, of pyridine derivatives, pyrimidine derivatives and pyrazole derivatives.

Among the pyridine derivatives mention may be made of compounds described for example in patents GB-A-1 026 978 and GB-A-1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine and their addition salts with an acid.

Other pyridine oxidation bases useful in the present invention are 3-aminopyrazolo[1,5-a]pyridine oxidation bases or their addition salts, described for example in patent application FR-A-2801308. By way of example mention may be made of pyrazolo[1,5-a]pyridin-3-ylamine; 2-acetylaminopyrazolo[1,5-a]pyridin-3-yl-amine; 2-morpholin-4-yl-pyrazolo[1,5-a]pyridin-3-yl-amine; 3-aminopyrazolo[1,5-a]pyridin-2-carboxylic acid; 2-methoxypyrazolo[1,5-a]pyridine-3-ylamino; (3-aminopyrazolo[1,5-a]pyridine-7-yl)methanol; 2-(3-aminopyrazolo[1,5-a]pyridine-5-yl)ethanol; 2-(3-amino-pyrazolo[1,5-a]pyridine-7-yl)ethanol; (3-amino-pyrazolo[1,5-a]pyridine-2-yl)methanol; 3,6-diamino-pyrazolo[1,5-a]pyridine; 3,4-diamino-pyrazolo[1,5-a]-pyridine; pyrazolo[1,5-a]pyridine-3,7-diamine; 7-morpholin-4-ylpyrazolo[1,5-a]pyridin-3-ylamine; pyrazolo-[1,5-a]pyridine-3,5-diamine; 5-morpholin-4-yl-pyrazolo-[1,5-a]pyridin-3-ylamine; 2-[(3-amino-pyrazolo[1,5-a]-pyridin-5-yl)-(2-hydroxyethyl)amino]ethanol; 2-[(3-aminopyrazolo[1,5-a]pyridin-7-yl)-(2-hydroxyethyl)-amino]ethanol; 3-amino-pyrazolo[1,5-a]pyridine-5-ol; 3-amino-pyrazolo[1,5-a]pyridine-4-ol; 3-amino-pyrazolo-[1,5-a]pyridine-6-ol; 3-aminopyrazolo[1,5-a]pyridine-7-ol; and their addition salts with an acid or with a base.

Among the pyrimidine derivatives mention may be made of the compounds described for example in patents DE-A-2359399; JP-A-88-169571; JP-A-05-63124; EP-A-0770375 or patent application WO-A-96/15765, such as 2,4,5,6-tetraminopyrimidine, 4-hydroxy-2,5,6-tri-aminopyrimidine, 2-hydroxy-4,5,6-triaminopyrimidine, 2,4-dihydroxy-5,6-diaminopyrimidine, 2,5,6-triamino-pyrimidine, and the pyrazolopyrimidine derivatives such as those mentioned in patent application FR-A-2750048 and among which mention may be made of pyrazolo[1,5-a]-pyrimidine-3,7-diamine; 2,5-dimethylpyrazolo[1,5-a]-pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-amino pyrazolo[1,5-a]pyrimidin-5-ol; 2-(3-aminopyrazolo-[1,5-a]pyrimidin-7-ylamino)ethanol, 2-(7-aminopyrazolo-[1,5-a]pyrimidin-3-ylamino)ethanol, 2-[(3-amino-pyrazolo[1,5-a]pyrimidin-7-yl)-(2-hydroxy-ethyl)amino]-ethanol, 2-[(7-aminopyrazolo[1,5-a]pyrimidin-3-yl)-(2-hydroxyethyl)amino]ethanol, 5,6-dimethylpyrazolo-[1,5-a]pyrimidine-3,7-diamine, 2,6-dimethylpyrazolo-[1,5-a]pyrimidine-3,7-diamine, 2,5,N7,N7-tetramethyl-pyrazolo[1,5-a]pyrimidine-3,7-diamine, 3-amino-5-methyl-7-imidazolylpropylaminopyrazolo[1,5-a]pyrimidine and their addition salts with an acid and their tautomeric forms, where a tautomeric equilibrium exists.

Among the pyrazole derivatives mention may be made of the compounds described in patents DE-A-3843892, DE-A-4133957 and patent applications WO-A-94/08969, WO-A-94/08970, FR-A-2 733 749 and DE-A-195 43 988, such as 4,5-diamino-1-methylpyrazole, 4,5-diamino-1-(β-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenyl-pyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-amino-ethyl)amino-1,3-dimethylpyrazole, 3,4,5-triamino-pyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and their addition salts with an acid.

Generally speaking the addition salts of the oxidation bases and of the couplers which can be used in the context of the invention are selected in particular from addition salts with an acid, such as hydrochlorides, hydrobromides, sulfates, citrates, succinates, tartrates, lactates, tosylates, benzene-sulfonates, phosphates and acetates, and addition salts with a base, such as sodium hydroxide, potassium hydroxide, ammonia, amines or alkanolamines.

The dyeing composition in accordance with the invention may further comprise one or more direct dyes which may in particular be selected from nitro dyes of the benzene series, direct azo dyes and direct methine dyes. These direct dyes may be nonionic, anionic or cationic in nature.

The medium appropriate for dyeing, also referred to as the dyeing vehicle, is generally composed of water or of a mixture of water and at least one organic solvent for dissolving the compounds which would not be sufficiently soluble in water. As an organic solvent mention may be made, for example, of C₁-C₄ alkanols, such as ethanol and isopropanol; polyols and polyol ethers, such as 2-butoxyethanol, propylene glycol, propylene glycol monomethyl ether, diethylene glycol monoethyl ether and monomethyl ether, and aromatic alcohols, such as benzyl alcohol or phenoxyethanol, and mixtures thereof.

The solvents are, preferably, present in proportions preferably between 1 and 40% by weight approximately, relative to the total weight of the dyeing composition, and more preferably still between 5 and 30% by weight approximately.

The dyeing composition in accordance with the invention may also include various adjuvants conventionally used in hair-dyeing compositions, such as anionic, cationic, nonionic, amphoteric and zwitterionic surfactants or mixtures thereof, anionic, cationic, nonionic, amphoteric or zwitterionic polymers or mixtures thereof, organic or inorganic thickeners, and especially anionic, cationic, nonionic and amphoteric polymer associative thickeners, antioxidants, penetrants, sequestrants, perfumes, buffers, dispersants, conditioning agents such as, for example, volatile or nonvolatile silicones, modified or unmodified, film formers, ceramides, preservatives and opacifiers.

The above adjuvants are generally present in an amount, for each of them, of between 0.01 and 20% by weight relative to the weight of the composition.

The skilled worker will of course make sure that this or these optional complementary compounds are selected such that the advantageous properties intrinsically attached to the oxidation dyeing composition in accordance with the invention are not, or not substantially, impaired by the addition or additions envisaged.

The pH of the dyeing composition in accordance with the invention is generally between 3 and 12 approximately, and preferably between 5 and 11 approximately. It may be adjusted to the desired value by means of acidifying and alkalifying agents which are customarily used in dyeing keratin fibers, or else by means of conventional buffer systems.

Among the acidifying agents mention may be made, by way of example, of organic or inorganic acids such as hydrochloric acid, ortho-phosphoric acid, sulfuric acid, carboxylic acids such as acetic acid, tartaric acid, citric acid and lactic acid, and sulfonic acids.

Among the alkalifying agents mention may be made, by way of example, of ammonia, alkali metal carbonates, alkanolamines such as mono-, di- and triethanolamines and their derivatives, sodium hydroxide or potassium hydroxide and the compounds of formula (A) below: 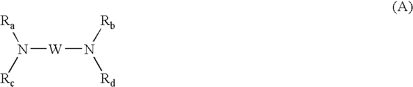
in which W is a propylene residue optionally substituted by a hydroxyl group or a C₁-C₄ alkyl radical; Rₐ, R_{b}, R_{c} and R_{d}, which are identical or different, represent a hydrogen atom or a C₁-C₄ alkyl or C₁-C₄ hydroxyalkyl radical.

The dyeing composition according to the invention may be provided in a variety of forms, such as in the form of liquids, creams or gels, or in any other form appropriate for carrying out dyeing of keratin fibers, more particularly human keratin fibers and especially human hair.

The method of the present invention is a method in which the composition according to the present invention as defined above is applied to the fibers and the color is developed by means of an oxidizing agent. The color may be developed at acidic, neutral or alkaline pH and the oxidizing agent may be added to the composition of the invention right at the time of use or it may be employed from an oxidizing composition comprising it, which is applied simultaneously or sequentially with the composition of the invention.

According to one particular embodiment, the composition according to the present invention is mixed, preferably at the time of use, with a composition comprising, in a medium appropriate for dyeing, at least one oxidizing agent, this oxidizing agent being present in an amount sufficient to develop a coloration. The resulting mixture is subsequently applied to the keratin fibers. After a time sufficient to develop the desired coloration, referred to as the leave-on time and amounting, for example, to 3 to 50 minutes approximately, preferably 5 to 30 minutes approximately, the keratin fibers are rinsed, washed with shampoo, rinsed again and then dried.

The oxidizing agents conventionally used for the oxidation dyeing of keratin fibers are, for example, hydrogen peroxide, urea peroxide, alkali metal bromates, persalts such as perborates and persulfates, peracids and oxidase enzymes, among which mention may be made of peroxidases, 2-electron oxidoreductases such as uricases and 4-electron oxygenases such as laccases. Hydrogen peroxide is particularly preferred.

The oxidizing composition may likewise include various adjuvants used conventionally in compositions for dyeing hair, and as defined above.

The pH of the oxidizing composition including the oxidizing agent is such that, after mixing with the dyeing composition, the pH of the resultant composition applied to the keratin fibers varies preferably between 3 and 12 approximately, and more preferably still between 5 and 11. It may be adjusted to the desired value by means of acidifying agents or alkalifying agents customarily used in dyeing keratin fibers, and as defined above.

The ready-to-use composition which is finally applied to the keratin fibers may be provided in a variety of forms, such as in the form of liquids, creams or gels or in any other form appropriate for carrying out dyeing of keratin fibers, and particularly of human hair.

The invention further provides a multi-compartment dyeing device or kit in which a first compartment includes, contains the dyeing composition of the present invention, defined above, and a second compartment includes, contains a composition with oxidizing agent. This device may be equipped with a means allowing the desired mixture to be delivered to the hair, such as the devices described in patent FR-A-2 586 913 in the name of the applicant.

By means of this device it is possible to dye the keratin fibers on the basis of a method which comprises mixing a dyeing composition comprising at least one oxidation base of formula (II), in the (R) or (S) configuration, with an oxidizing agent and applying the resulting mixture to the keratin fibers for a time sufficient to develop the desired coloration.

The invention will now be described with reference to the following example, which is given by way of illustration and not of limitation.

### EXAMPLE 1

(3R)-1-(4-Nitrophenyl)pyrrolidin-3-ol, compound of formula (I) ((I_{R})), in which R₁ is hydrogen, is prepared by the process according to the invention in its first variant, in accordance with scheme 4 below:

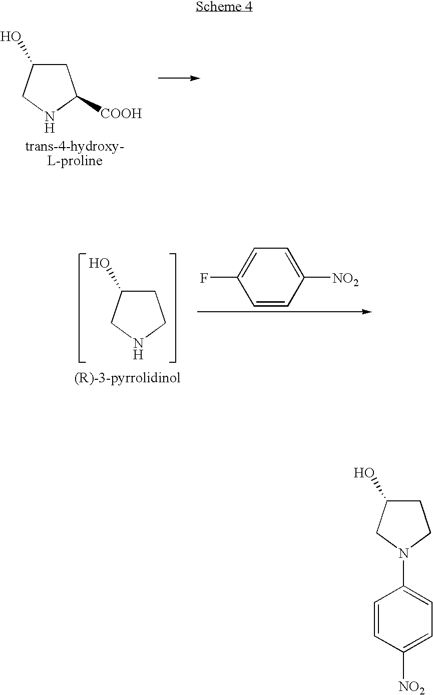

In a reactor, under a nitrogen atmosphere, 100 g (0.763 moles) of trans 4-hydroxy-L-proline are introduced in 500 ml of N-methylpyrrolidone and 5 ml of cyclohexene-1-one.

The mixture is heated at 155° C. for 3 h and then cooled to the ambient temperature.

126.5 g (0.915 moles) of potassium carbonate are introduced and then 96.8 g (0.686 moles) of 4-fluoro-1-nitrobenzene in solution in 125 ml of N-methyl-pyrrolidone are run into the reactor. The mixture is heated at 105° C. for 3 hours. It is cooled to 10° C. and then poured into a large volume of iced water. The precipitate is filtered off. It is washed with water and then dried under vacuum at 50° C. This gives 127.5 g of a yellow solid, equivalent to a final yield of 89%. The solid obtained is characterized by the following analyses.

Analyses

The mass spectrum and 1H and 13C NMR spectra are in accordance with the expected structure.

Melting point (DSC): 180.7° C.

Elemental analysis (C12H16N202: MW=220.27).

| | C | H | N | O |
|---|---|---|---|---|
| % theoretical | 57.69 | 5.81 | 13.45 | 23.05 |
| % found | 57.55 | 5.82 | 13.46 | 22.84 |

1-[(3S)-1-(4-Aminophenyl)pyrrolidin-3-yl]-3-methyl-1H-imidazol-3-ium chloride, hydrochloride, compound of formula (II) ((II_{S})), in which R₁ is hydrogen and R₂ represents a cationic nitrogen-containing radical, is prepared by the process according to the following scheme: 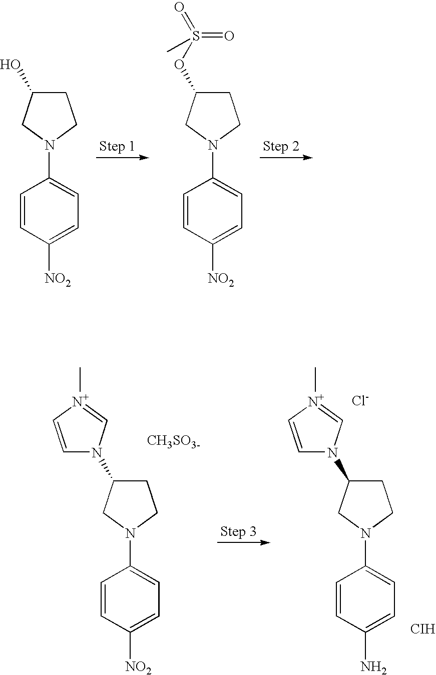
Step 1:

### (3R)-1-(4-nitrophenyl)pyrrolidin-3-yl methanesulfonate

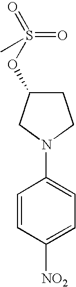

In a reactor, under a nitrogen atmosphere, 75.8 g (0.364 moles) of the compound from the preceding step are introduced in 580 ml of pyridine. The mixture is cooled to 0° C. and then 33.9 ml (0.437 moles) of methanesulfonyl chloride are added, while the temperature is maintained at below 5° C.

The mixture is stirred at 5° C. for 3 h 45 min and then poured into 5.25 L of water.

The mixture is filtered and the solid is rinsed with water and then dried under vacuum at 40° C.

This gives 79 g of a pinkish beige solid, equivalent to a final yield of 76%.

Analyses

The mass spectrum and 1H and ¹³C NMR spectra are in accordance with the expected structure.

Step 2:

### 3-methyl-1-[(3S)-1-(4-nitrophenyl)pyrrolidin-3-yl]-1H-imidazol-3-ium methanesulfonate

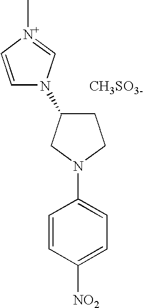

In a reactor, under a nitrogen atmosphere, 200 g (0.67 moles) of the preceding compound are introduced in 2000 ml of methyl isobutyl ketone. The mixture is taken to 80° C. and then 211 ml of N-methylimidazole are added dropwise. The mixture is subsequently heated at reflux at 115° C. for 6 h, cooled to 20° C. and then filtered.

The solid is washed with acetone and then dried under vacuum.

This gives 188.3 g of a light-beige solid, equivalent to a yield of 73%.

Analyses

The mass spectrum and 1H and 13C NMR spectra are in accordance with the expected structure.

Step 3:

### 1-[(3S)-1-(4-Aminophenyl)pyrrolidin-3-yl]-3-methyl-1H-imidazol-3-ium chloride, hydrochloride

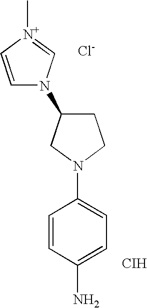

In a stainless-steel hydrogenation reactor 12 g (32.57 mmol) of the compound from the preceding step are partially dissolved in 200 ml of 96 ethanol. 1.2 g of 5% Pd/C (50% moist) are added, the reactor is closed and purged with nitrogen 3 times with stirring (1800 rpm). Hydrogen is subsequently introduced under a pressure of 5-6 bars at ambient temperature. The temperature rises to 31° C. and then, after 1 h 15 min, falls back to 27° C.

The reactor is then purged with nitrogen and the reaction mixture is filtered under a nitrogen atmosphere. The filtrate is recovered immediately in a solution containing 13.5 ml of 37% hydrochloric acid and 20 ml of 96% ethanol.

The filtrate is subsequently evaporated until a precipitate is obtained.

The solid is filtered off, washed with isopropanol and then with ethyl ether and dried under vacuum in the presence of potassium hydroxide.

This gives 5.1 g of a white solid, equivalent to a yield of 49%.

1. A process for preparing an optically active 1-(4-nitrophenyl)-3-pyrrolidinol derivative corresponding to formula (I) below: 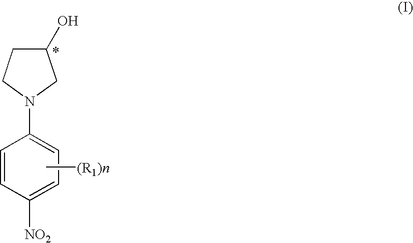
in which:
the carbon bearing the OH substituent on the pyrrolidine ring is chiral, and possesses the (R) or (S) configuration;
n is an integer of 0 to 4, with the proviso that when n is greater than or equal to 2 the radicals R₁ may be identical or different;
R₁ represents a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl moiety is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ not containing a peroxide linkage nor diazo, nitro or nitroso radicals;
wherein the following successive steps are carried out:
a) trans-4-hydroxy-L-proline is decarboxylated: 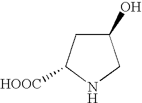
to give (R)-3-pyrrolidinol: 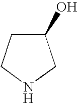
b) without isolation, the resulting (R)-3-pyrrolidinol formed is reacted with a 4-halonitrobenzene of formula (V): 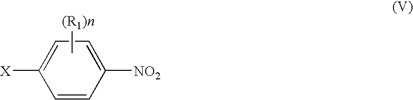
in which X represents an F, Cl, Br or I atom and R₁ is as already defined above for the formula (I), thereby giving the compound of formula (I) in which the carbon bearing the —OH substituent on the pyrrolidine ring possesses the (R) configuration (compound I_{R}): 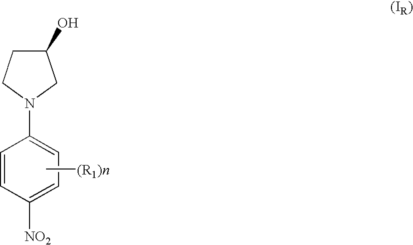
c) optionally the configuration of the compound of formula (I_{R}) is inverted, thereby giving the compound of formula (I_{S}):

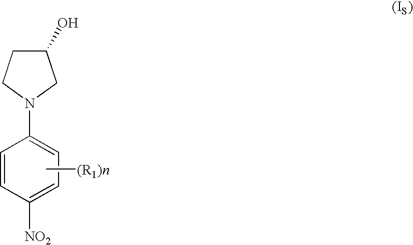

2. A process for preparing an optically active 1-(4-nitrophenyl)-3-pyrrolidinol derivative of formula (I): 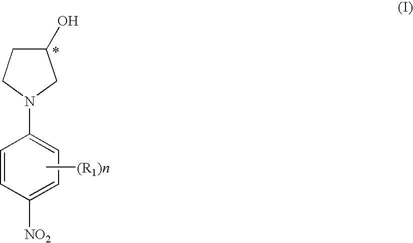
in which:
the carbon bearing the OH substituent on the pyrrolidine ring is chiral, and possesses the (R) or (S) configuration;
n is an integer of 0 to 4, with the proviso that when n is greater than or equal to 2 the radicals R₁ may be identical or different;
R₁ represents a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl moiety is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ not containing a peroxide linkage or diazo, nitro nor nitroso radicals;
wherein the following successive steps are carried out:
a′) trans-4-hydroxy-L-proline: 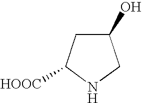
is condensed with a 4-halonitrobenzene of formula (V): 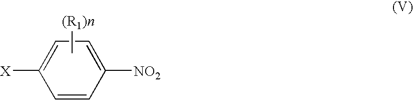
in which X represents an F, Cl, Br or I atom and R₁ is as already defined above for the formula (I), to give a compound of formula (VI): 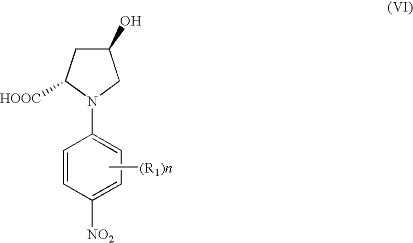
b′) the compound of formula (VI) is decarboxylated to give the compound of formula (I) in which the carbon bearing the OH substituent on the pyrrolidine ring possesses the (R) configuration (compound I_{R}): 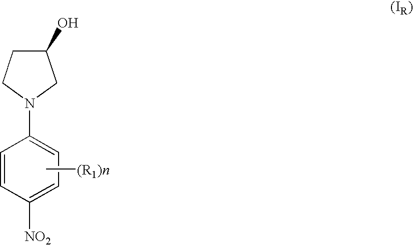
c′) optionally the configuration of the compound of formula (I_{R}) is inverted, thereby giving a compound of formula (I_{S}):

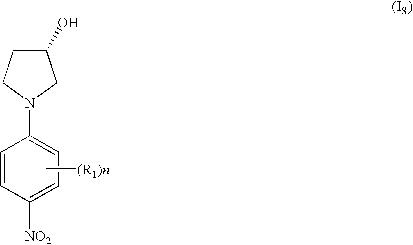

3. The process as claimed in claim 1 or 2, wherein n is 0.

4. The process as claimed in claim 1 or 2, n is other than 0 and is for example 1, and R₁ is a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl chain is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ containing no peroxide linkage and no diazo, nitro or nitroso radicals.

5. The process as claimed in any one of claims 1, 2 and 4, wherein R₁ is selected from chlorine, bromine, a C₁-C₆, advantageously C₁-C₄, alkyl radical, a (C₁-C₄)alkoxy-(C₁-C₄) alkyl radical, a C₁-C₄ hydroxyalkoxy, C₁-C₄ alkoxy or C₁-C₄ hydroxyalkoxy radical.

6. The process as claimed in claim 5, wherein R₁ is selected from methyl, hydroxymethyl, 2-hydroxyethyl, 1,2-dihydroxyethyl, methoxy, isopropyl-oxy and 2-hydroxyethoxy radicals.

7. The process as claimed in claim 1 or 2, wherein the inversion of configuration of the compound of formula (I_{R}) in step c) or c′) is carried out by performing a Mitsunobu reaction on the compound of formula (I_{R}) to give a compound of formula (VII): 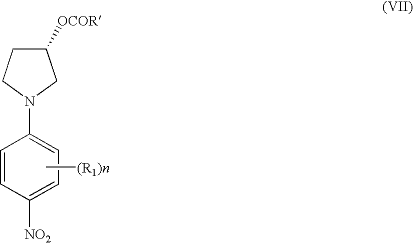
in which R₁ and n are as already defined in claim 1 or claim 2 and R′ represents a hydrogen atom, a C₁-C₆ hydrocarbon radical or a C₆-C₈ aryl radical, then by hydrolyzing the compound of formula (VII) to give the compound of formula (I_{S}).

8. Para-Phenylenediamine derivatives substituted by a chiral pyrrolidinyl group, of formula (II), obtainable from an intermediate derivative of formula (I) as described in claim 1, and their addition salts: 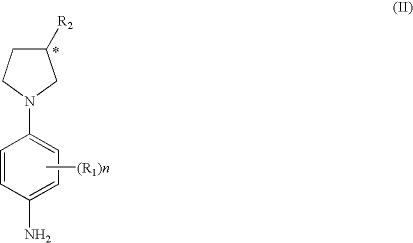
in which:
the carbon atom which bears the substituent R₂ on the pyrrolidine ring is chiral and possesses the (R) or (S) configuration;
n represents an integer from 0 to 4, with the proviso that when n is greater than or equal to 2 the radicals R₁ can be identical or different;
R₁ represents a halogen atom; a saturated or unsaturated, C₃-C₈ alicyclic, or C₁-C₈ preferably C₁-C₆, linear or branched, aliphaticn hydrocarbon chain, an aryl radical, an arylalkyl radical whose alkyl moiety is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ not containing a peroxide linkage nor diazo, nitro or nitroso radicals;
R₂ represents a cationic or noncationic nitrogen-containing radical.

9. Derivatives as claimed in claim 8 wherein n is 0.

10. Derivatives as claimed in claim 8, wherein n is other than 0 and is for example 1, and R₁ is a halogen atom; a saturated or unsaturated, C₃-C₈ alicyclic, or C₁-C₈ preferably C₁-C₆, linear or branched, aliphatic, hydrocarbon chain, an aryl radical, an arylalkyl radical whose alkyl chain is C₁-C₈, preferably C₁-C₆, it being possible for one or more carbon atom(s) of the hydrocarbon chain and of the alkyl chain of the arylalkyl radical to be replaced by an oxygen, nitrogen, silicon or sulfur atom or by an SO₂ group; the radical R₁ not containing a peroxide linkage nor diazo, nitro or nitroso radicals.

11. Derivatives as claimed in claim 10, wherein R₁ is selected from chlorine, bromine, a C₁-C₆, advantageously C₁-C₄, alkyl radical, a C₁-C₄ hydroxyalkyl radical, a (C₁-C₄)alkoxy-(C₁-C₄) alkyl radical, a C₁-C₄ alkoxy radical or a C₁-C₄ hydroxyalkoxy radical.

12. Derivatives as claimed in claim 11, wherein R₁ is selected from methyl, hydroxymethyl, 2-hydroxyethyl, 1,2-dihydroxyethyl, methoxy, isopropyl-oxy and 2-hydroxyethoxy radicals.

13. Derivatives as claimed in any one of claims 8 to 12, wherein R₂ is a noncationic nitrogen-containing radical and represents a primary (—NH₂), secondary (—NHR) or tertiary (—NR₂) amine radical in which the RS, which are identical or different, represent a saturated or unsaturated, linear or branched C₁-C₂₂ aliphatic radical, preferably a C₁-C₂₂ alkyl radical; a saturated or unsaturated C₃-C₈ alicyclic radical; a C₁-C₂₂, preferably C₁-C₆, monohydroxyalkyl radical; a C₂-C₂₂, preferably C₂-C₆, polyhydroxyalkyl radical; a (C₁-C₆) alkoxy-(C₁-C₂₂, preferably C₁-C₆)-alkyl radical; an aryl radical; an arylalkyl radical whose alkyl moiety is C₁-C₆, such as the benzyl radical, for example; an amido(C₁-C₆) alkyl radical; a tri-(C₁-C₆)alkyl-silane-(C₁-C₆) alkyl radical; a C₁-C₆ aminoalkyl radical; or a C₁-C₆ aminoalkyl radical whose amine is mono- or di-substituted by a C₁-C₄ alkyl, (C₁-C₆)alkyl-carbonyl, amido or (C₁-C₆)alkyl-sulfonyl radical or the radicals R may form in pairs, together with the nitrogen atom to which they are attached, a saturated carbon ring containing 3 to 9 members, preferably 4, 5, 6, 7 or 8 members, which may contain one or more heteroatoms; examples of such rings are azetidine, pyrrolidine, piperidine, piperazine, morpholine rings, it being possible for said heterocycle to be substituted by a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri-(C₁-C₆)alkyl-silane-(C₁-C₆) alkyl radical, an amido radical, a carboxyl radical, a (C₁-C₆)alkyl-carbonyl radical, a thio radical (—SH), a C₁-C₆ thioalkyl radical (—R—SH), a (C₁-C₆)alkyl-thio radical, an amino radical, an amino radical mono- or di-substituted by a (C₁-C₆)alkyl, (C₁-C₆)alkyl-carbonyl, amido or (C₁-C₆)alkyl-sulfonyl radical.

14. Derivatives as claimed in any one of claims 8 to 12, wherein R₂ is a radical derived from aminoguanidine.

15. Derivatives as claimed in any one of claims 8 to 12, wherein R₂ represents an onium radical Z corresponding to the formula (VIII): 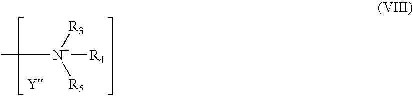
R₃, R₄ and R₅, taken separately, identical or different, represent a saturated or unsaturated, linear or branched C₁-C₂₂ aliphatic radical, preferably a C₁-C₂₂ alkyl radical; a saturated or unsaturated C₃-C₈ alicyclic radical; a C₁-C₂₂, preferably C₁-C₆, monohydroxyalkyl radical; a C₂-C₂₂, preferably C₂-C₆, polyhydroxyalkyl radical; a (C₁-C₆)alkoxy-(C₁-C₂₂, preferably C₁-C₆) alkyl radical; an aryl radical; an arylalkyl radical whose alkyl moiety is C₁-C₆, such as the benzyl radical, for example; a amido (C₁-C₆) alkyl radical; a C₁-C₆ tri-(C₁-C₆) alkyl-silane-(C₁-C₆) alkyl radical; a C₁-C₆ aminoalkyl radical; or a C₁-C₆ aminoalkyl radical whose amine is mono- or di-substituted by a C₁-C₄ alkyl, (C₁-C₆)alkyl-carbonyl, amido or (C₁-C₆)alkyl-sulfonyl radical; or
R₃, R₄ and R₅ together, in pairs, form, with the nitrogen atom to which they are attached, a saturated carbon ring containing 3 to 9 members, preferably 4, 5, 6, 7 or 8 members, which may contain one or more heteroatoms; as examples of this ring mention may be made of azetidine, pyrrolidine, piperidine, piperazine or morpholine rings, it being possible for said cationic heterocycle to be substituted by a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri-(C₁-C₆)alkylsilane-(C₁-C₆) alkyl radical, an amido radical, a carboxyl radical, a (C₁-C₆)alkyl-carbonyl radical, a thio radical (—SH), a C₁-C₆ thioalkyl radical (—R—SH), a (C₁-C₆)alkyl-thio radical, an amino radical, or an amino radical mono- or di-substituted by a (C₁-C₆)alkyl, (C₁-C₆)alkyl-carbonyl, amido or (C₁-C₆)alkyl-sulfonyl radical;
Y″ is a counterion.

16. Derivatives as claimed in claim 15, wherein in the formula (VIII) R₃, R₄ and R₅ are selected separately from a C₁-C₆ alkyl radical, a C₁-C₄ monohydroxyalkyl radical, a C₂-C₄ polyhydroxyalkyl radical, a (C₁-C₆)alkoxy-(C₁-C₄) alkyl radical, a C₁-C₆ amidoalkyl radical, a tri-(C₁-C₆)alkyl-silane-(C₁-C₆) alkyl radical, or R₃ together with R₄ forms an azetidine ring, a pyrrolidine, piperidine, piperazine or morpholine ring, R₅ being selected in this case from a C₁-C₆ alkyl radical; a C₁-C₆ monohydroxy alkyl radical; a C₂-C₆ polyhydroxyalkyl radical; a C₁-C₆ aminoalkyl radical, an aminoalkyl radical mono- or di-substituted by a (C₁-C₆)alkyl, (C₁-C₆)alkyl-carbonyl, amido or (C₁-C₆)alkyl-sulfonyl radical; a carbamyl (C₁-C₆) alkyl radical; a tri-(C₁-C₆)alkyl-silane-(C₁-C₆) alkyl radical; a (C₁-C₆)alkyl-carboxy (C₁-C₆) alkyl radical; a (C₁-C₆)alkyl-carbonyl (C₁-C₆) alkyl radical; or a N—(C₁-C₆)alkyl-carbamyl (C₁-C₆) alkyl radical.

17. Derivatives as claimed in claim 15 or 16, wherein in the formula (VIII) the radical R₂ is a trialkylammonium radical whose alkyl radicals may be substituted.

18. Para-phenylene derivatives as claimed in any one of claims 8 to 12, wherein R₂ represents an onium radical Z corresponding to the formula (IX): 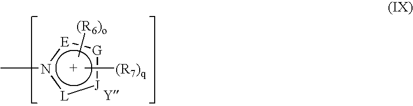
in which:
the ring members E, G, J and L, which are identical or different, represent a carbon, oxygen, sulfur or nitrogen atom, to form a pyrrole, pyrazole, imidazole, triazole, oxazole, isooxazole, thiazole or isothiazole ring;
q is an integer between 0 and 4 inclusive;
o is an integer between 0 and 3 inclusive;
q+o is an integer between 0 and 4 inclusive;
R₇s, which are identical or different, represent a hydroxyl radical, a C₁-C₆ alkyl radical, a C₁-C₆ monohydroxyalkyl radical, a C₂-C₆ polyhydroxyalkyl radical, a C₁-C₆ alkoxy radical, or a tri(C₁-C₆)alkyl-silane-(C₁-C₆) alkyl radical; with the proviso that the radicals R₇ are borne by a carbon atom;
R₆, identical or different, represents a C₁-C₆ alkyl radical, a C₁-C₆ monohydroxyalkyl radical, a C₂-C₆ polyhydroxyalkyl radical, a tri(C₁-C₆)alkyl-silane-(C₁-C₆) alkyl radical, a (C₁-C₆) alkoxy-(C₁-C₆) alkyl radical or a benzyl radical; with the proviso that the radicals R₆ are borne by a nitrogen;
Y″ is a counterion.

19. Derivatives as claimed in claim 18, wherein the ring members E, G, J and L form a pyrrole, imidazole, pyrazole, oxazole, thiazole or triazole ring.

20. Derivatives as claimed in claim 19, wherein the ring members E, G, J and L form an imidazole ring.

21. para-Phenylenediamine derivatives according to any one of claims 8 to 12, wherein R₂ represents the onium radical Z corresponding to the formula (X): 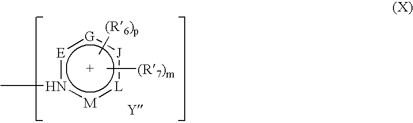
in which:
the ring members E, G, J, L and M, which are identical or different, represent a carbon, oxygen, sulfur or nitrogen atom, to form a ring selected from pyridine, pyrimidine, pyrazine, triazine and pyridazine rings;
p is an integer between 0 and 3 inclusive;
m is an integer between 0 and 5 inclusive;
p+m is an integer between 0 and 5 inclusive;
R′₇s, which are identical or different, represent a hydroxyl radical, a C₁-C₆ alkyl radical, a C₁-C₆ monohydroxyalkyl radical, a C₂-C₆ polyhydroxyalkyl radical, a C₁-C₆ alkoxy radical, or a tri(C₁-C₆)alkyl-silane-(C₁-C₆) alkyl radical; with the proviso that the radicals R′₇ are borne by a carbon atom;
R′₆, identical or different, represents a C₁-C₆ alkyl radical, a C₁-C₆ monohydroxyalkyl radical, a C₂-C₆ polyhydroxyalkyl radical, a tri(C₁-C₆)alkyl-silane-(C₁-C₆) alkyl radical, a (C₁-C₆) alkoxy-(C₁-C₆) alkyl radical or a benzyl radical; with the proviso that the radicals R′₆ are borne by a nitrogen;
Y″ is a counterion.

22. Derivatives as claimed in claim 21, wherein the ring members E, G, J, L and M with the nitrogen of the ring form a pyridine or pyrimidine ring.

23. Derivatives as claimed in any one of claims 18 to 22, wherein R₆, R₇, R′₇ and R′₆ are alkyl radicals, which can be substituted.

24. A dyeing composition comprising, in a medium appropriate for dyeing keratin fibers, at least one para-phenylenediamine of formula (II), in an (R) or (S) configuration, as defined in any one of claims 8 to 23, as oxidation base.

25. The composition as claimed in claim 24, further comprising at least one coupler selected from meta-phenylenediamines, meta-aminophenols, meta-diphenols, naphthalenic couplers, heterocyclic couplers and their addition salts.

26. The composition as claimed in any one of claims 24 or 25, further comprising at least one additional oxidation base other than the oxidation bases of formula (II), selected from para-phenylenediamines, bis-phenylalkylenediamines, para-aminophenols, ortho-aminophenols, heterocyclic bases and their addition salts.

27. The composition as claimed in any one of claims 24 to 26, wherein the amount of oxidation base of formula (II) is between 0.001 and 10%, preferably between 0.005 and 6% by weight approximately of the total weight of the dyeing composition.

28. The composition as claimed in any one of claims 24 to 27, wherein the amount of each of the additional oxidation bases is between 0.001 and 10% by weight approximately of the total weight of the dyeing composition.

29. The composition as claimed in any one of claims 24 to 28, wherein the amount of each of the couplers is between 0.001 and 10%, preferably between 0.005 and 6% by weight approximately of the total weight of the dyeing composition.

30. A method of oxidation-dyeing keratin fibers, characterized in that a dyeing composition as defined in any one of claims 24 to 29 is applied to the fibers in the presence of an oxidizing agent for a time sufficient to develop the desired coloration.

31. The method as claimed in claim 30, wherein the oxidizing agent is selected from hydrogen peroxide, urea peroxide, alkali metal bromates, persalts, peracids and oxidase enzymes.

32. A multicompartment device in which a first compartment contains a dyeing composition as defined in any one of claims 24 to 29 and a second compartment contains an oxidizing agent.

33. The use of the composition defined in claims 24 to 29 for dyeing keratin fibers.
